(19) United States
(12) Patent Application Publication
(10) Pub. No.: US 2025/0312138 A1
(43) Pub. Date: Oct. 9, 2025

(54) AORTIC VALVES AND RELATED METHODS

(71) Applicant: inQB8 Medical Technologies, LLC, Burlington, MA (US)

(72) Inventors: Arshad Quadri, West Hartford, CT (US); J. Brent Ratz, Burlington, MA (US); Garrett Johnson, Costa Mesa, CA (US); Levi John Nicholas Caffes, Somerville, MA (US); Caroline Elizabeth Buckley, Belmont, MA (US); Jack Berkman Sattell, Cambridge, MA (US)

(73) Assignee: inQB8 Medical Technologies, LLC, Burlington, MA (US)

(21) Appl. No.: 19/170,348

(22) Filed: Apr. 4, 2025

Related U.S. Application Data
(60) Provisional application No. 63/575,371, filed on Apr. 5, 2024.

(51) Int. Cl. A61F 2/07; A61F 2/00; A61F 2/24

(52) U.S. Cl. CPC A61F 2/07; A61F 2/2418; A61F 2002/0081; A61F 2210/0076; A61F 2250/001; A61F 2250/0024

(57) ABSTRACT

The present disclosure generally relates to implantable medical devices, and, in some embodiments, to a prosthetic aortic implant. Such implantable devices may be useful in the treatment of acute aortic dissections (AADs). related systems and methods are also provided. In some embodiments, the prosthetic aortic implant comprises an expandable support structure and at least a first portion sized and configured to be positioned within an ascending portion of a native aorta of a subject. In some embodiments, the first portion comprises a non-porous layer and a porous layer. In some embodiments, the first portion is sized and configured to engage an aortic root of the native aorta. In some embodiments, the prosthetic aortic implant is designed and configured to engage with and/or receive an aortic valve implant.

### RELATED APPLICATIONS

This application claims the benefit of U.S. Provisional Patent Application Ser. No. 63/575,371, filed Apr. 5, 2024, entitled “AORTIC VALVES AND RELATED METHODS,” which is incorporated herein by reference in its entirety for all purposes.

### TECHNICAL FIELD

The present invention generally relates to implantable medical devices, and, more particularly, to prosthetic aortic implants, as well as systems and methods involving the same. Such devices, systems, and methods may be useful for e.g., the treatment of Acute Aortic Dissections (AAD), Intramural Hematomas and Thoracic Aortic Aneurysms.

### BACKGROUND

Management of AADs depend on the type of dissection and its location along the aorta, but generally involves medications, to reduce heart rate and lower blood pressure which help to prevent the ADD from worsening, and/or surgery, to remove as much of the dissected aorta as possible and to stop blood from leaking into the aortic wall. However, nearly 10-30% of all AADs are deemed inoperable and managed primarily with medication alone. The mortality in this population is high, with approximately 15-30% of patients dying within 24 hrs, which tapers off to approximately 1% per day from day 6 through day 30. Outcomes for surgical candidates are equally poor with sequela rates, e.g., mortality and neurological damage, as high as 15-30%. Accordingly, improved devices and methods are needed.

### SUMMARY

The present invention generally relates to implantable medical devices, and, more particularly, to a prosthetic aortic implant, systems comprising the prosthetic aortic implant, and related methods. The subject matter of the present disclosure involves, in some cases, interrelated products, alternative solutions to a particular problem, and/or a plurality of different uses of one or more systems and/or articles.

Aspects of the present disclosure generally relate to a prosthetic aortic implant. In some embodiments, the prosthetic aortic implant comprises an expandable support structure having a first portion sized and configured to be positioned within an ascending portion of a native aorta, and a second portion, wherein the first portion is configured to apply radial force to an aortic root of the aorta when expanded. In some embodiments, the first portion comprise a non-porous layer adjacent a first porous layer and configured to contact an outer wall of the native aorta. In some embodiments, an expandable anchoring structure is located at a proximal end of the first portion sized and configured to engage an aortic root of the native aorta. In some embodiments, the second portion comprises a second porous layer. In some embodiments, the expandable anchoring structure comprises one or more backstop elements sized and configured to engage a native leaflet of the native aorta. In some embodiments, the proximal end of the first portion is sized and configured to receive an aortic valve implant.

Some aspects of the disclosure generally relate to systems. In some embodiments, the systems comprise a prosthetic aortic valve and a prosthetic aortic implant comprising a proximal end sized and configured to receive the prosthetic aortic valve. In some embodiments, the prosthetic aortic implant comprises a first portion sized and configured to be positioned within an ascending portion of a native aorta, one or more expandable anchoring structures, and a second portion sized and configured to be positioned within a descending portion of the native aorta. In some embodiments, the expandable anchoring structure is sized and configured to apply radial force to an aortic root of the aorta when expanded.

In another aspect, the present disclosure generally encompasses methods of making one or more of the embodiments described herein, for example, prosthetic aortic implant. In still another aspect, the present disclosure encompasses methods of using one or more of the embodiments described herein, for example, prosthetic aortic implant.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIGS. 1A-C illustrates various components of a first porous layer (FIG. 1A), a nonporous layer (FIG. 1B) and a first portion (FIG. 1C) of a prosthetic aortic implant, according to some embodiments;

FIGS. 2A-B illustrate various components of a second portion (FIG. 2A) and a prosthetic aortic implant (FIG. 2B); according to some embodiments;

FIG. 3 illustrates a prosthetic aortic implant positioned within a native aorta; according to some embodiments;

FIG. 4 illustrates a first portion of a prosthetic aortic implant comprising a prosthetic aortic valve frame; according to some embodiments;

FIGS. 5A-5B illustrates a proximal end of a first portion of a prosthetic aortic implant comprising a prosthetic aortic valve frame positioned within a native aortic root, wherein the valve frame is not engaged with a prosthetic aortic valve implant. FIG. 5B shows a same configuration with a prosthetic aortic valve implant inserted within the valve frame; according to some embodiments,

FIGS. 6A-B illustrates a prosthetic aortic implant comprising a prosthetic aortic valve frame positioned within a native aorta. FIG. 6B shows the same configuration with a prosthetic aortic valve implant inserted within the valve frame, according to some embodiments.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

The present disclosure generally relates to implantable medical devices, and, in some embodiments, to a prosthetic aortic implant. Such implantable devices may be useful in the treatment of acute aortic dissections (AADs). related systems and methods are also provided. In some embodiments, the prosthetic aortic implant comprises an expandable support structure and at least a first portion sized and configured to be positioned within an ascending portion of a native aorta of a subject. In some embodiments, the first portion comprises a non-porous layer and a porous layer. In some embodiments, the first portion is sized and configured to engage an aortic root of the native aorta. In some embodiments, the prosthetic aortic implant is designed and configured to engage with and/or receive an aortic valve implant.

The prosthetic aortic implants may be implanted (e.g., surgically) in a subject to a subject to treat a disease, disorder, or other clinically recognized condition, or for prophylactic purposes, and/or may have a clinically significant effect on the body of the subject to treat and/or prevent the disease, disorder, or condition. A “subject” refers to any animal such as a mammal (e.g., a human). Non-limiting examples of suitable subjects include a human, a non-human primate, a cow, a horse, a pig, a sheep, a goat, a dog, a cat or a rodent such as a mouse, a rat, a hamster, a bird, a fish, or a guinea pig. Generally, the invention is directed toward use with humans. In some embodiments, a subject may demonstrate health benefits, e.g., upon implantation of the prosthetic aortic implant.

In some embodiments, the prosthetic aortic devices disclosed herein are useful for the treatment of subjects suffering from one or more types of Acute Aortic Dissections (AADs).

As would be understood by those of ordinary skill in the art, AADs generally occur when a portion of the aortic intima (the inner most layer of the aorta) ruptures and systemic blood pressure serves to delaminate the intimal layer from the media layer resulting in a false lumen for blood flow that can propagate in multiple directions along the length of the aorta. AADs that occur in the ascending portion of the aorta may generally be classified as Acute Type A Aortic Dissections (ATAADs, also referred to as Type 1 and Type 2 according to De Bakey classification system), whereas those not involving the ascending aorta are referred to as Type B dissections (according to the Stanford classification system). In some cases, failure to rapidly treat AADs, and particularly, ATAADs, may lead to severe sequela including stroke, organ damage, e.g., kidney failure or life-threatening intestinal damage, aortic valve damage, and death due to severe internal bleed (e.g., mortality rate is nearly 50% at 48 hours post injury and 90% within 30 days post injury).

Those of ordinary skill will understand, based upon the teachings of this specification that the systems, methods, and devices described herein may, in some embodiments, fill an important therapeutic gap in the treatment of patients with AADs. For example, the prosthetic aortic implants described herein may advantageously be useful for providing a prophylactic that may be administered non-invasively in an outpatient setting. In some embodiments, the prosthetic aortic implants described herein may advantageously be administered to patients with recently diagnosed aortic aneurysms, for example, as a preventative measure to delay (or prevent) disease progression. In other embodiments, the prosthetic aortic implants may advantageously be useful for rapidly treating patients suffering from ATAADs (e.g., an aortic dissection in the ascending aorta that occur acutely and rapidly without warning, as may occur in patients with undiagnosed aortic aneurysms). In some embodiments, placement (e.g., implantation) of the prosthetic aortic implant within the ascending aorta of a patient suffering from ATAAD may serve to reinforce the inner wall of the aorta near the dissection and re-establish a true lumen for blood to flow through. In some embodiments, the prosthetic aortic implants described herein may advantageously provide a non-invasive method to fix damaged aortic valves, for example, by incorporating a valve frame configured to reversibly (or irreversibly) receive a prosthetic aortic valve. For example, in some embodiments, the prosthetic aortic implants described herein may be sized and configured to receive (e.g., reversibly) a transcatheter aortic valve implant (TAVI). In some embodiments, an aortic valve such as a TAVI is positioned within the prosthetic aortic implant and/or a portion of the native aorta that has been configured to receive the TAVI as a result of the presence of the prosthetic aortic implant.

The prosthetic aortic implants described herein may have several advantages over previously described devices. For example, some previously described devices generally comprise a short one-piece implant constructed of fabric with built-in reinforcements configured to reside within the ascending aorta alone. However, such devices may be prone to movement and dislocation e.g., because they generally lack features that may anchor the device to the native aorta. In contrast to traditional devices, the prosthetic aortic implants described herein comprise, in some embodiments, one or more expandable anchoring structures, configured to engage and apply a radial outward force, to one or more structures of a native aorta, e.g., aortic sinuses and/or the sinotubular junction, within an aortic root of the native aorta, thus anchoring the device to the native aorta (e.g., reducing the likelihood of movement and/or dislocation). In some embodiments, the disclosed devices are configured to extend from the ascending aorta into the descending aorta, wherein the descending portion further anchors the device to the native aorta, e.g., advantageously further reducing the likelihood of movement and/or dislocation.

In some cases, aortic grafts for treating aortic aneurysms may be used to treat ATAADs, wherein the aortic grafts generally comprise a non-porous layer to wall off the aneurysm from the main lumen of the graft and aorta. However, such grafts cannot generally be placed over regions of the aorta (e.g., the aortic arch) e.g., that require fenestration windows so blood may flow to branched vessels. The prosthetic aortic implants described herein may advantageously comprise, in some embodiments, a porous layer positioned over at least part of an expandable support structure, e.g., thereby permitting the graft to span from the ascending aorta into the descending aorta without blocking blood flow to critical branch vessels (e.g., brachiocephalic artery, left common carotid artery, and the left subclavian artery).

In some cases, bare-metal implants have also been described for the treatment of AADs. However, bare-metal frames are generally abrasive and may erode through the tissue and/or cause the fragile intima layer to dissect further. The prosthetic aortic implants described herein may advantageously comprise, in some embodiments, an expandable reinforcement structure comprising an atraumatic outer layer configured to distribute a radial force throughout the entire aorta, e.g., thereby reducing the likelihood of the aneurysms rupturing.

In some embodiments, the prosthetic aortic implant comprises an expandable support structure comprising a first portion configured to be positioned within an ascending portion of a native aorta. In some embodiments, one or more expandable anchoring structures located at a proximal end of the first portion may be sized and configured to engage one or more structures of a native aorta, e.g., aortic sinuses and/or the sinotubular junction, within an aortic root of the native aorta. In some embodiments, the expandable support structure is sized and configured to apply a radially outward force, e.g., to anchor the first portion to the native aorta. In some cases, the first portion comprises a nonporous layer. In some embodiments, the nonporous layer comprises an outer surface (e.g., the outer surface being atraumatic to the native aorta). In some embodiments, the nonporous layer is adjacent a porous layer (e.g., the porous layer provided over the expandable support structure). In some embodiments, the porous layer is configured to contact an inner wall of the ascending aorta e.g., adjacent a false lumen associated with the dissection. The nonporous layer may, in some cases, be configured to expand. Without wishing to be bound by theory, the nonporous layer may expand, in some cases, due to blood hydrostatic pressure created by blood flowing through the intraluminal space formed between the non-porous layer and the first porous layer. In some cases, upon expanding, the non-porous layer applies a radially outward force to the ascending aorta, e.g., advantageously preventing blood flow through the dissection.

In some embodiments, the expandable support structure comprises a second portion sized and configured to be positioned within a descending portion of the native aorta, wherein a proximal end of the second portion is in contact with a distal end of the first portion, such that the second portion further anchors the first portion to the native aorta. The second portion, in some cases, may comprise a second porous layer, for example, to permit blood flow from within the expandable support structure, through the second porous layer, and into the carotid arteries and/or subclavian arteries of the native aorta. In some embodiments, a system for treating AADs is provided, wherein the system comprises an expandable support structure and a prosthetic aortic valve frame, positioned at the proximal end of the first portion, wherein the prosthetic aortic valve frame is configured to receive a valve, e.g., a bridge valve or a destination valve.

Some aspects of the expandable support structure, porous layer, and nonporous layer are described in U.S. Pat. No. 10,888,414, entitled “Aortic Dissection Implant”, and filed on Mar. 19, 2020, which is incorporated herein by reference in its entirety for all purposes.

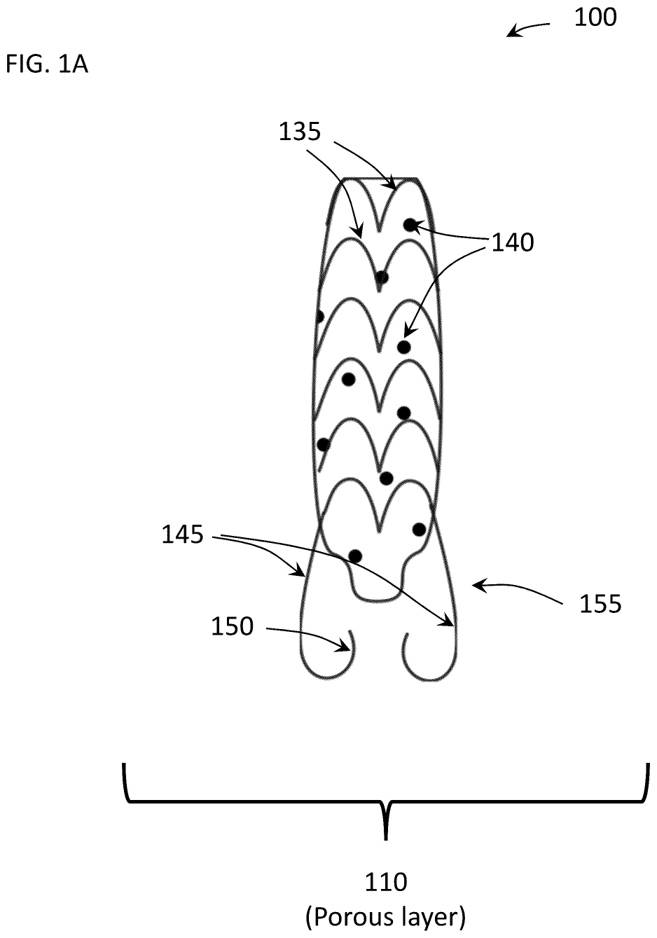

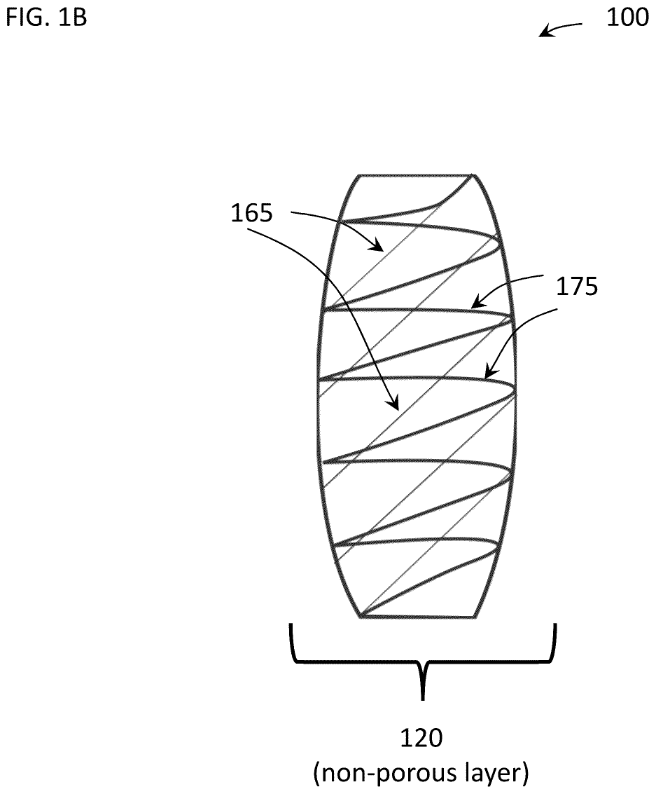

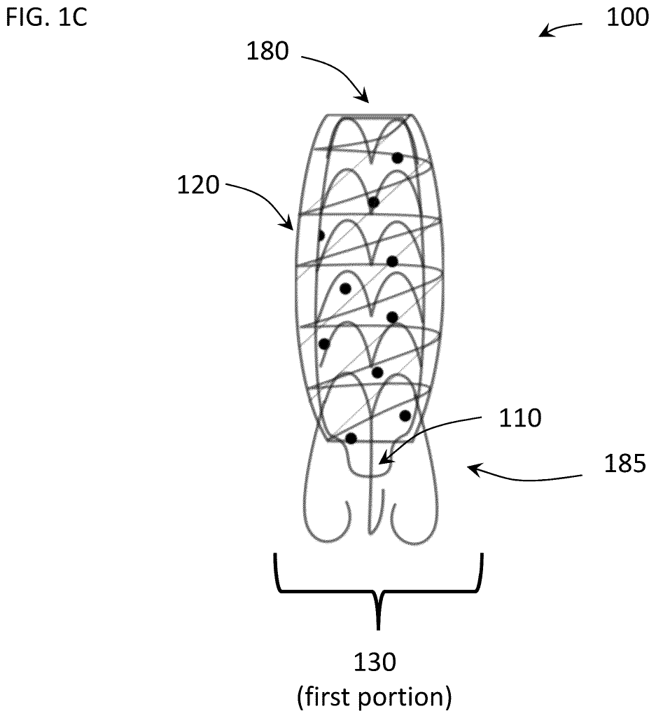

FIG. 1A shows an embodiment of exemplary device 100 comprising a first porous layer 110, configured to be positioned within an ascending portion of a native aorta. First porous layer 110 comprises a porous material 140 (e.g., a porous fabric or polymer membrane) positioned over a first expandable reinforcement structure 135 (e.g., a wire or coil). In some embodiments, first porous layer 110 further comprises one or more expandable anchoring structures 145, configured to be positioned within an aortic root of the native aorta. In some embodiments, expanding anchoring structure 145 comprises one or more backstop elements 150 located at a proximal end 155 of the first porous layer 110. FIB. 1B shows another embodiment of exemplary device 100, comprising a non-porous layer 120, which may also be positioned with an ascending portion of the native aorta. In the embodiment shown, non-porous layer 120 comprises a non-porous material 165 (e.g., a non-porous fabric or polymer membrane) positioned over a second expandable reinforcement structure 175 (e.g., a wire or coil). FIG. 1C, shows another embodiment of exemplary device 100 wherein porous layer 110 is positioned within non-porous layer 120 thereby forming first portion 130. The configuration shown in FIG. 1C, according to some embodiments, places the non-porous layer in contact with an inner wall of the native aorta and adjacent to first porous layer 110.

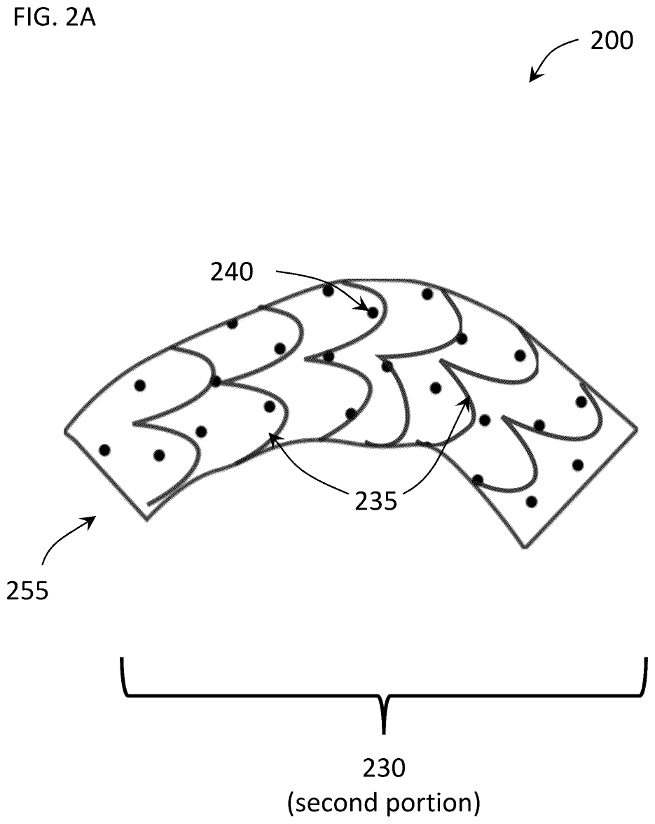

FIG. 2A shows an embodiment of exemplary device 200 comprising a second portion 230 configured to be positioned within a descending portion of a native aorta. Second portion 230 may comprise a second porous layer comprising a second porous material 240 positioned over a third expandable reinforcement structure 235. FIG. 2B shows an embodiment of exemplary device 200, comprising a first portion 130 and second portion 230, wherein a proximal end 255 of second portion 230 is configured to engage a distal end 180 of first portion 130.

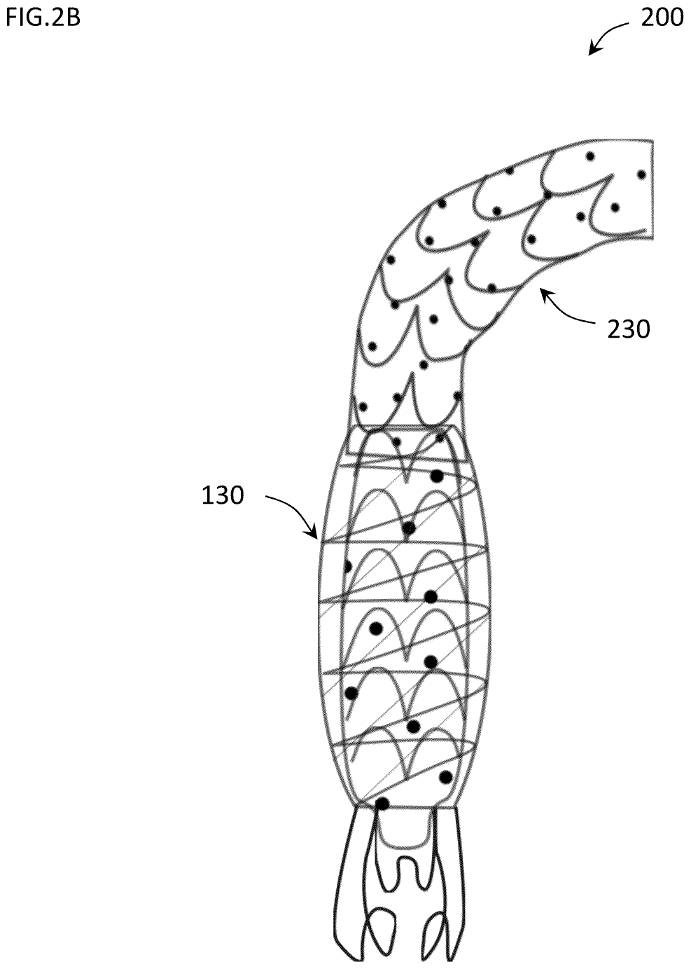

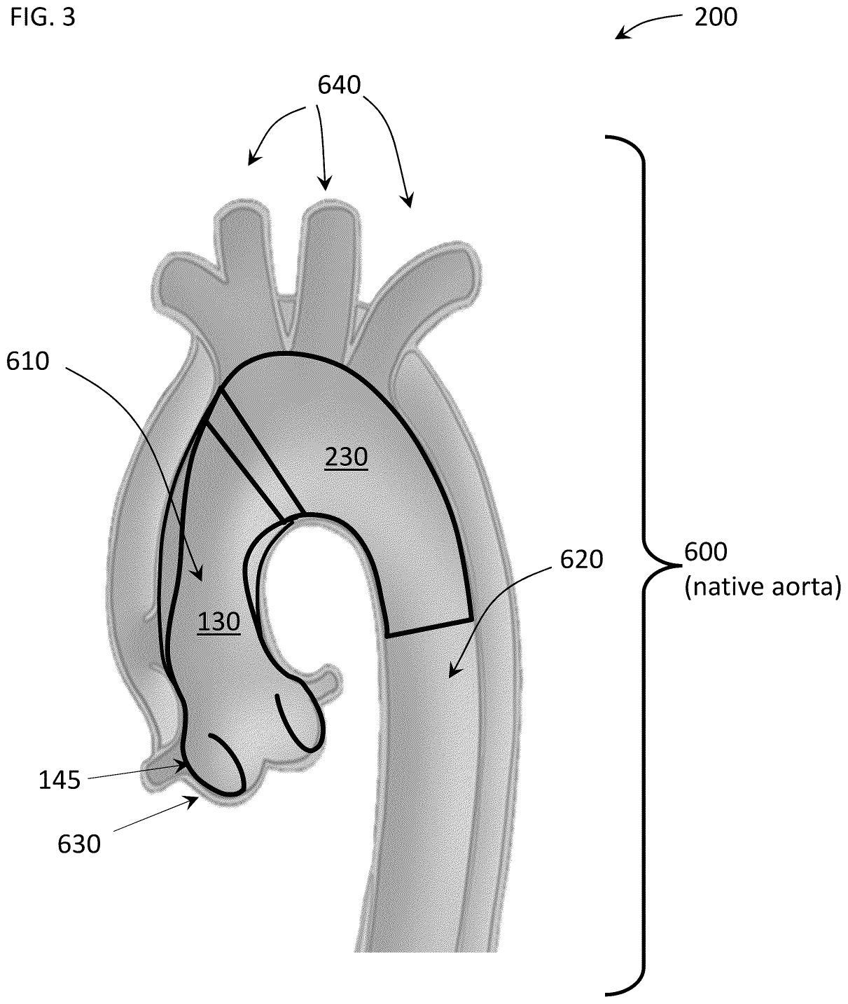

FIG. 3 shows an embodiment of exemplary device 200 positioned within native aorta 600, wherein exemplary device 200 comprises a first portion 130, configured to be positioned with an ascending portion 610 of the native aorta 600, and a second portion 230, configured to be positioned within a descending portion 620 of the native aorta 600. Expandable anchoring structures 145 may be positioned within an aortic root and adjacent to an aortic sinus 630 of native aorta 600. Those of ordinary skill in the art will understand that such configurations may exert a radial outward force that anchors the first portion 130 to native aorta 600. In some embodiments, the expandable anchoring structures may comprise a sinusoidal structure positioned within the aortic valve and configured to apply a radially outward force to a sinotubular junction (not shown for clarity purposes). The second portion 230 may be configured to overlap the first portion and to extend past the branched vessels of the aortic arch 640. Such configurations may allow blood to flow unobstructed from the device into the branched vessels of the aortic arch 640 while simultaneously preventing first portion 130 from migrating into descending portion 620 of the native aorta 600. (e.g., the second portion may further act to anchor the device to the native aorta).

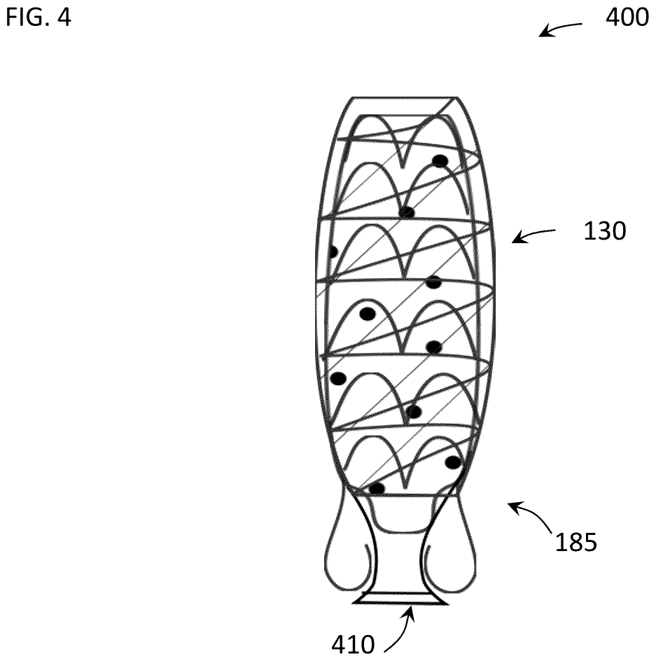

FIG. 4 shows an embodiment of exemplary device 400, wherein first portion 130 further comprises a prosthetic aortic valve 410 located at proximal end 185 of first portion 130, wherein the valve frame is configured to receive (either reversibly or irreversibly) a prosthetic aortic valve. Such designs may take advantage of the anchoring features (e.g., expandable anchoring structures and backstops) of first portion 130, while also providing the ability to reversibly (or irreversibly) administer a prosthetic aortic valve implant, for example, using minimally invasive techniques (e.g., percutaneous implantation) at any point in time. This may be advantageous, for example, in the treatment of patients presenting with aortic valve regurgitation at the time of implantation or develop regurgitation because of disease progression.

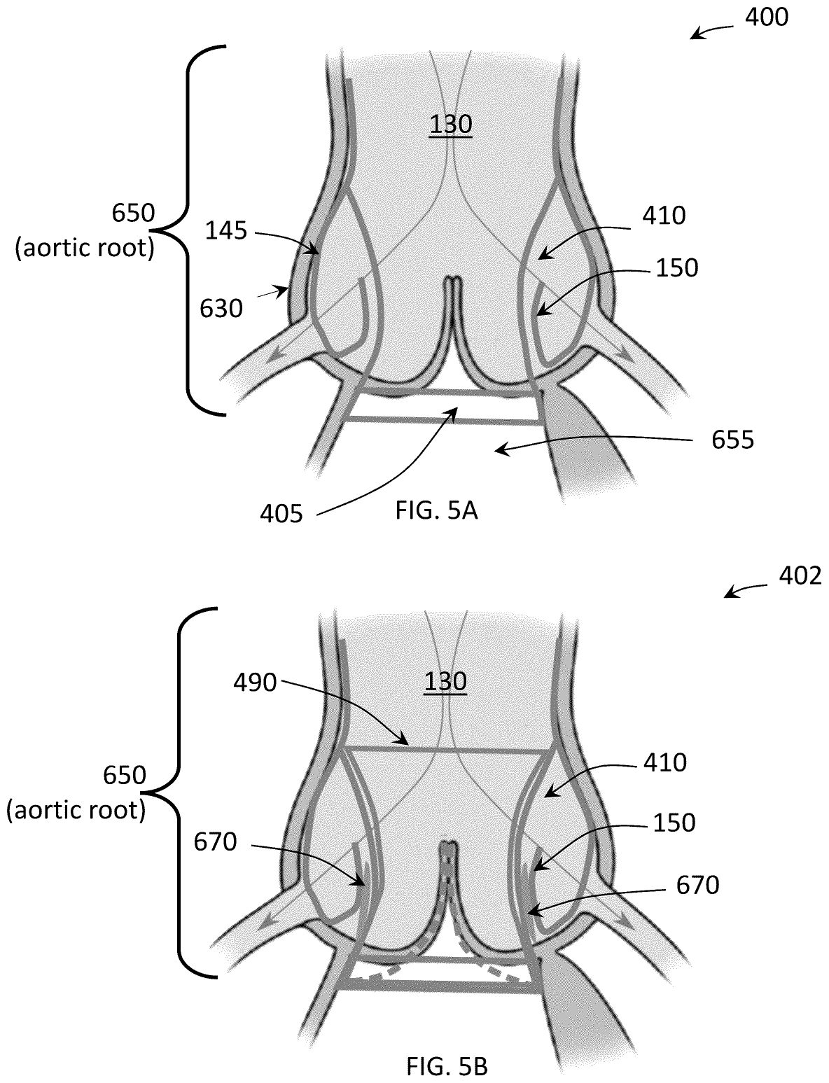

FIG. 5A shows an embodiment of exemplary device 400 positioned within aortic root 650 of a native aorta. In the current embodiment, first portion 130 comprises one or more expandable anchoring structures 145 comprising one or more backstop elements 150 and prosthetic aortic valve frame 410 that extends from proximal end 185 of the first portion 130 into left ventricular outflow track (LVOT) 655. In some embodiments, proximal end 405 of the valve frame 410 (e.g., the part that extends into the LVOT) may be flared, thus providing an additional mechanism for anchoring the device to the native aorta. FIG. 5B shows an exemplary embodiment of device 402 comprising a first portion 130 and prosthetic aortic valve frame 410, wherein the valve frame comprises a prosthetic aortic valve 490. In some embodiments, placing prosthetic aortic valve 490 into prosthetic aortic valve frame 410 pushes one or more native leaflets 670 up against the one or more backstop elements 150. In some embodiments, such a configuration may advantageously prevent the leaflets from obstructing blood flow into the coronary arteries. In some embodiments, such a configuration anchors device 402 to aortic root 650.

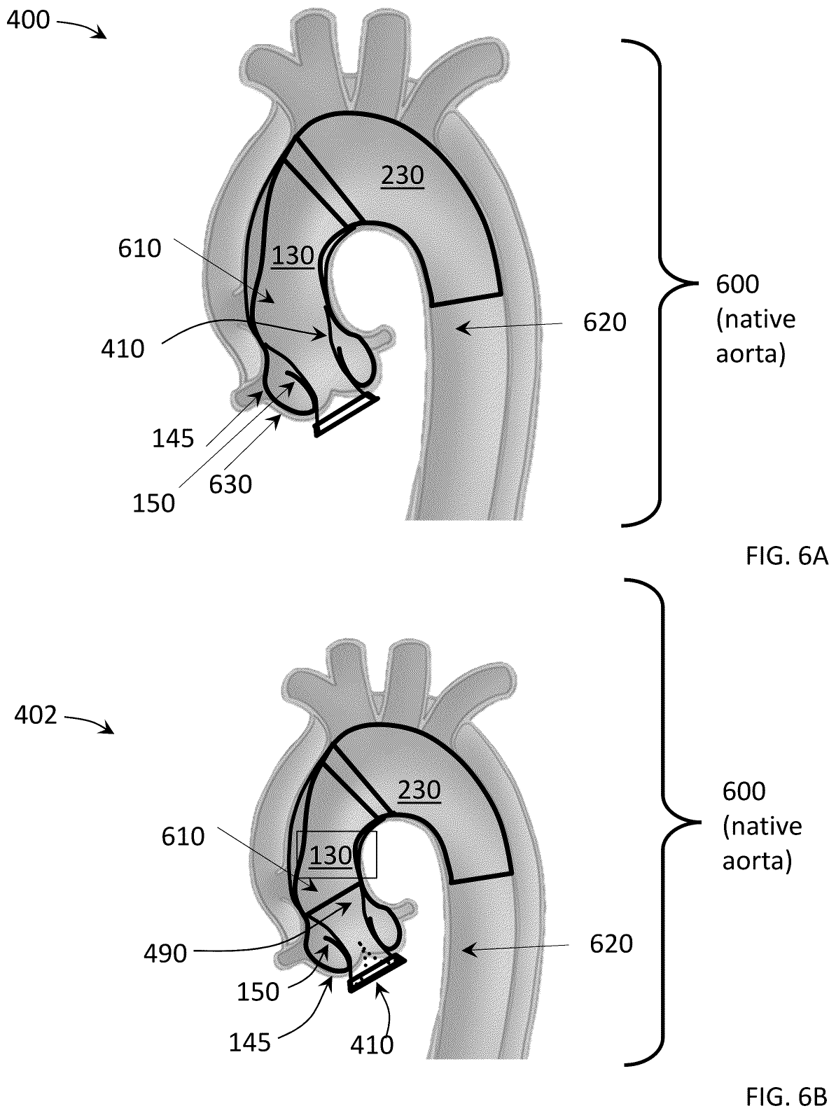

FIG. 6A shows an embodiment of exemplary device 400, comprising a first portion 130 and a second portion 230 positioned within native aorta 600 in a manner similar to that described in FIG. 3. In the current exemplary embodiments, first portion 130 comprises prosthetic aortic valve frame 410, one or more expandable anchoring structures 145, and one or more backstop elements 150. Proximal end 405 of prosthetic aortic valve frame 410 extends past the aortic root and into the left ventricular outflow track; and the one or more expandable anchoring structures 145 rests within the aortic root and may apply a radial outward force against one or more aortic root sinuses 630. Second portion 230 comprises a porous layer comprising a porous material positioned over an expandable reinforcement structure. In an exemplary embodiment, second portion 230 may be positioned under the branched vessels of the aortic arch and configured to overlap first portion 130, such that the device extends from ascending portion 610 of native aorta 600 to descending portion 620 of native aorta 600. FIG. 6B shows an exemplary embodiment of device 402, comprising device 400 and prosthetic aortic valve 490, positioned with prosthetic aortic valve frame 410. In some embodiments, the one or more expandable anchoring structures 150 and/or the extension of the graft into the descending aorta may advantageously serve to anchor the implant to native aorta 600 to prevent movement and dislocation. In some embodiments, the one or more backstop elements 150 may preserve blood flow to the coronary sinuses by preventing the implantable prosthetic aortic valve from pressing the one or more native leaflets into coronary ostium. In some embodiments, each backstop of the one or more backstops 150 are configured to engage one or more native leaflets of a native heart valve.

In some embodiments, a prosthetic aortic implant comprises an expandable support structure comprising one or more frames (e.g., support structures), configured to provide the overall structure of the implant. For example, in some embodiments, the prosthetic aortic implant may comprise a first portion comprising a prosthetic aortic valve frame. In some embodiments, the prosthetic aortic frame may be configured to receive an aortic valve implant, either during (or after) deployment of the device. In some embodiments, the first portion comprises a proximal end, wherein the proximal end is positioned within an aortic root of a native aorta and is sized and configured to receive an aortic valve implant. In some embodiments, the proximal end of the first portion comprises a prosthetic aortic valve frame, configured to receive the aortic valve implant, for example, an implant with a tri-leaflet design. In some embodiments, a proximal end of the prosthetic aortic valve frame is flared and extends into a left ventricular outflow tract (LVOT). In some embodiments, placing the flared proximal end within the LVOT anchors the device to the native aorta, thus reducing the likelihood of undesired movement and dislocation.

In some embodiments, the prosthetic aortic valve frame may comprise a “bridge” valve, for example, to serve as a temporary valve (e.g., <24 hrs). In other embodiments, a permanent valve, e.g., a commercially available TAVR (transcatheter aortic valve replacement), may be placed within the bridge valve between about 24 hours to 48 hours post deployment of the prosthetic aortic implant, for example, using a non-invasive percutaneous approach. In some embodiments, the prosthetic aortic valve frame may comprise a destination valve, for example, as a permanent aortic valve replacement option. In some embodiments, the prosthetic aortic valve frame may be configured to reversibly (or irreversibly) receive the aortic valve implant. The ability to repeatedly remove the valve, for example, during percutaneous placement of a TAVR may permit optimal fitting of the prosthetic within the bridge valve. In some embodiments, the prosthetic aortic valve may comprise a tri-lobe (or tri-leaflet) design (e.g. to mimic the native aortic valve) and may comprise a bioprosthetic material (e.g., porcine or bovine aortic valves) or a synthetic material (e.g., dacron or the like).

In some embodiments, an expandable support structure may comprise one or more frames (e.g., support structures) may comprising one or more expandable anchoring structures configured, for example, to anchor the device to the native aorta while preserving blood flow, thus decreasing likelihood of undesired movement and possible dislocation and/or vascular obstruction following initial deployment of the device. In some embodiments, the one or more expandable anchoring structures of a first portion may comprise a metallic wire frame; in other embodiments, the one or more expandable anchoring structures may be integrated into other frames (e.g., valve frame and/or reinforcement structures).

In some embodiments, the one or more expandable anchoring structures are located at a proximal end of the first portion, wherein the one or more expandable anchoring structures are configured to engage at least one structure within an aortic root of a native aorta. For example, in some embodiments, the expandable anchoring structures may extend within a left and/or right aortic valve sinus without obstructing the coronary ostia (e.g., the left and right coronary ostia remain uncovered such that blood flow into the coronaries is unaffected).

Such configurations may permit the one or more expandable anchoring structures to apply radially outward forces against one or more aortic sinuses (e.g., Sinus of Valsalva) and/or to promote a seal at within the region just above the sinotubular junction in the native aorta. In some embodiments, the expandable anchoring structures may be covered, for example, by a first porous layer. In such configurations, the one or more expandable anchoring structures, configured to extend within a left and a right aortic valve sinus, may be at least partially uncovered such that the left and right coronary ostia remain uncovered (e.g., coronary blood flow is preserved) by the aortic dissection implant when in use.

In some embodiments, one or more expandable anchoring structures may comprise an expandable trilobe structure and/or an expandable sinusoidal structure. In some embodiments, the expandable trilobe structure may comprise a trilobe structure comprising three lobes, wherein each of the three lobes is sized and configured to conform to a curvature of the aortic valve sinus when the expanded anchoring structure is in an expanded configuration. In some embodiments, each of the three lobes comprises one or more apices at a distal end of an expandable trilobe structure configured to be positioned adjacent to an aortic valve annulus of the patient. In some embodiments, at least a portion of the expandable trilobe structure applies a radially outward force to an aortic sinus thereby anchoring the prosthetic aortic implant to the native aorta. In some embodiments, the expandable sinusoidal structure may apply a radially outward force to a sinotubular junction thereby anchoring the prosthetic aortic implant to the native aorta.

Those of ordinary skill in the art will appreciate that placement of an prosthetic aortic valve within a prosthetic aortic valve frame may push one or more native leaflets of a native aortic valve against the coronary ostium and may obstruct (either partially or completely) blood flow to the coronary vessels (e.g., by blocking either the left and/or right coronary arteries). Therefore, in some embodiments, the one or more expandable anchoring structures may comprise one or more backstop elements sized and configured to engage a native leaflet of the native aorta, for example, during placement of a prosthetic valve, thus preventing the native leaflets from expanding beyond the backstops (e.g., preserving coronary blood flow). In some embodiments, the one or more backstop elements may have a particular radius of curvature.

In some embodiments, an expandable support structure comprises one or more frames comprising one or more expandable reinforcement structures configured, for example, to expand within a native aorta and to distribute a radial load (e.g., induced by hydrostatic blood pressure) at least partially across the native aorta, thus reducing the pressure applied to the fragile aortic wall. In some embodiments, the one or more expandable reinforcement structure may be a first expandable reinforcement structure, a second expandable reinforcement structure, a third expandable reinforcement structure, a fourth expandable reinforcement structure, etc.

In some embodiments, one or more frames (e.g., aortic valve frames, expanding anchoring structures, and/or expanding reinforcement structures) may comprise a naturally derived textile (e.g., silk, collagen, elastin, Rayon, and the like) and/or a synthetically derived textile (e.g., medical grade metals, alloys, and polymers). Exemplary medical grade metals include titanium, tantalum, copper and brass, nitinol (e.g., nickel-titanium alloy), stainless steel, cobalt-chrome alloy, gold, platinum, silver, iridium, tantalum, tungsten, etc. Exemplary medical grade polymers include acrylonitrile butadiene styrene (ABS), acetal copolymer, acetal copolymer, acetal homopolymer (Delrin), polyethylene terephthalate polyester (PET-P), polytetrafluoroethylene (Fluorosint), ethylene-chlorotrifluoro-ethylene (Halar), polybutylene terephthalate-polyester (Hydex), polyvinylidene fluoride (Kynar), polyphenylene oxide (Noryl), nylon, polyetheretherketone (PEEK), polycarbonate, polyethylenes (LDPE, HDPE, and UHMW), polypropylene homopolymer, polyphenylsulfone (PPSU), polysulfone (PSU), polyethersulfone (Radel A), polyarylethersulfone (Radel R), Rulon 641. Any other suitable material may also be used to produce the one or more frame structures (e.g., aortic valve frames, expanding anchoring structures, and/or expanding reinforcement structures).

In some embodiments, one or more frames (e.g., aortic valve frames, expanding anchoring structures, and/or expanding reinforcement structures) may be formed into any desired geometry (e.g., tubular, flared, etc.) using any technique known to those of skill in the art. In some embodiments, the expandable reinforcement structures may be formed by weaving, for example, metallic fibers and/or polymeric fibers, into yarns and/or fabrics that may be used to create the frame structures. As defined herein, a fiber refers to a textile (either natural or synthetic) processed into a threadlike strand (e.g., metallic fibers, polymer fibers, etc); yarn refers to a plurality of fibers (e.g., biologic, polymeric, and/or metallic) that are twisted together (or otherwise entangled) to improve strength, abrasion resistance, and handling of the fibers; whereas fabrics refer to yarns that are interlaced by various mechanical processes (e.g., weaving, knitting, and braiding).

Natural fibers, (e.g., collagen, silk, elastin, etc.), metallic fibers, and polymeric fibers may be processed into yarns and fabrics, using techniques known to those of skill in the art, to create complex three-dimensional shapes (e.g., tubular geometries with tapered angles, etc.). For example, in some embodiments, the frames (e.g., aortic valve frames, expanding anchoring structures, and/or expanding reinforcement structures) may be a woven structure, in which two sets of yarns are interlaced at right angles; in other embodiments, the frames (e.g., aortic valve frames, expanding anchoring structures, and/or expanding reinforcement structures) may be a knit structure, in which loops of yarn are intermeshed; and in some cases, the frames (e.g., aortic valve frames, expanding anchoring structures, and/or expanding reinforcement structure) may be braided, in which three or more yarns cross one another in a diagonal pattern, according to other embodiments.

As will be appreciated by those of skill in the art, one or more frames (e.g. aortic valve frames, expanding anchoring structures, and/or expanding reinforcement structures) comprising knit fabrics may be either weft or warp knit, and braided products may include tubular structures, with or without a core, as well as ribbons. In some embodiments, the frames (aortic valve frames, expanding anchoring structures, and/or expanding reinforcement structures) comprising woven fabrics may be stitched using a technique known as a Leno weave, to avoid unraveling at the edges when cut squarely or obliquely, as may be performed by a surgeon, for example, during deployment of the device. In some embodiments, a combination of fibers may be used to create the one or more frame structures (e.g., aortic valve frames, expanding anchoring structures, and/or expanding reinforcement structures). For example, in some embodiments, a combination of fibers may be oriented both helically and axially, which may provide high strength with excellent elasticity. In other embodiments, fibers may be oriented helically and axially, wherein the helical fibers are interlocked into a circular shape around axially aligned fibers. Such configurations may enable production of thin-walled structures (e.g., tubes) with high strength.

In some embodiments, one or more frames (e.g., aortic valve frames, expanding anchoring structures, and/or expanding reinforcement structures) may be formed using a nonwoven material (e.g., a natural and/or synthetic material). Nonwovens may be made directly from natural and/or synthetic fibers (e.g., polymeric fibers) that are needle-felted, hydroentangled, or bonded through a thermal, chemical, and/or adhesive process. In other embodiments, nonwoven structures (e.g., frames) may also be made directly from a polymer, such as the polymers listed elsewhere herein. For example, in some embodiments, the one or more expandable reinforcement structures may be formed using electrostatically spun polyurethane to produce porous tubular structures. In other embodiments, still, the one or more frame structures may be formed by laser cutting various patterns into preformed structures, such as tubular structures. Other techniques are also possible, for example, metals and/or polymer may be extruded into thin wires that may be knit, woven, and/or braided into any of the aforementioned structures (e.g., tubes)

In some embodiments, a prosthetic aortic implant may comprise various combinations of frames (e.g., aortic valve frames, expanding anchoring structures, and/or expanding reinforcement structures). For example, in some embodiments, a first portion may comprise a first expandable reinforcement structure and one or more expandable anchoring structures. The first portion may comprise a first expandable reinforcement structure that extends continuously from a proximal end to a distal end of the first portion and that is continuous with one or more expandable anchoring structures (e.g. they are the same frame). In some embodiments, the first portion and the expandable anchoring structure are formed from a single continuous wire (e.g., a metallic wire and/or polymeric wire). In some embodiments, the expandable reinforcement structure and the expandable anchoring structure are both metallic. In other embodiments, the expandable reinforcement structure and the expandable anchoring structure are both polymeric. In other embodiments, still, the expandable reinforcement structure and the expandable anchoring structure comprise a combination of polymeric and/or metallic materials. In some embodiments, the first portion and the expandable anchoring structures are separate structures (e.g., separate frames)

In some embodiments, a prosthetic aortic implant comprises a first portion, comprising a first expandable reinforcement structure (e.g., frame) and one or more expandable anchoring structures, and a second portion comprising a second expandable reinforcement structure (e.g., a frame). In some embodiments, the second portion comprises a second expandable reinforcement structure (e.g., a frame) that extends continuously from a proximal end to a distal end of the second portion and that is continuous with the expandable anchoring structure (e.g., they are the same frame). In some embodiments, the second portion and the one or more expandable anchoring structures comprise separate structures (e.g., separate frames). In some embodiments, the first portion, the expandable anchoring structure, and the second portion comprise separate structures (e.g., three separate frames). In other embodiments, the first portion, the expandable anchoring structure, and second portion comprise the same structure (e.g. same continuous frame). In some embodiments, the first portion, the expandable anchoring frame and the second portion are formed from a single continuous wire (e.g., a metallic and/or polymeric wire).

In some embodiments, a prosthetic aortic implant comprising one or more frames may be deployed synchronously (e.g., at the same time) or asynchronously (e.g., staged deployment). For example, in some embodiments, a first portion comprising an expandable anchoring structure comprising a trilobe structure (or sinusoidal structure) may be deployed asynchronously, such that the trilobe structure (or sinusoidal structure) is configured to expand and engage an inner wall of the aortic sinus separately from expansion of the first portion (e.g., first and/or second expandable structures), and the prosthetic aortic implant is configured to sequentially deploy the trilobe structure (or the sinusoidal structure) before (or after) the first portion. In other embodiments, the trilobe structure (or sinusoidal structure) may be configured to expand and engage the inner wall of the aortic sinus simultaneity with the first portion and the prosthetic aortic implant is configured to simultaneously deploy the trilobe structure (or the sinusoidal structure) and the first portion.

In some embodiments, a prosthetic aortic implant comprises an expandable support structure comprising a porous layer. As used herein, porous refers to any material and/or structure with at least a portion of pores sufficiently large enough to allow blood to flow through. In some embodiments, the porous layer (e.g., first porous layer, second porous layer, etc.) is the same as the frame structure. For example, in some embodiments, the first expandable reinforcement structure is the first porous layer; and the third expandable reinforcement structure is the second porous layer. In other embodiments, a porous material (e.g., woven nylon) may be provided over, within, or embedded inside the frame structure (e.g., the first expandable reinforcement structure). Any suitable porous material may be used, such as nonwoven, woven, knit, and/or braided fabrics (e.g., natural and/or artificial fabrics). In other embodiments, the porous material may comprise a polymeric film with a plurality of pores, wherein the pores are formed, for example, using chemical processes (e.g., phase separation) and/or physical processes (e.g., laser etched).

In some embodiments, the porosity of a porous layer may vary around the circumference and/or along the length of the porous layer. For example, in some embodiments, the porosity of the frame structure (e.g., first, second, and/or third expandable reinforcement structures) may vary around the circumference and/or along the length of the frame structure (e.g., first, second, and/or third expandable reinforcement structures.

In some embodiments, the average pore size of a first porous layer may be between about 0.01 mm and 2 mm. In some embodiments, the average pore size of a first porous layer is greater than or equal to 0.01 mm, greater than or equal to 0.05 mm, greater than or equal to 0.1 mm, greater than or equal to 0.5 mm, greater than or equal to 0.75 mm, greater than or equal to 1 mm, greater than or equal to 1.5 mm, and greater than or equal to 2.0 mm. In some embodiments, the average pore size is less than or equal to 2.0 mm, less than or equal to 1.5 mm, less than or equal to 1 mm, less than or equal to 0.75 mm, less than or equal to 0.5 mm, less than or equal to 0.1 mm, less than or equal to 0.05 mm, and less than or equal to 0.01 mm. Combinations of the above-referenced ranges are also possible (e.g., greater than or equal to 0.01 mm and less than or equal to 2 mm). Other ranges are also possible.

In some embodiments, the average thickness of the first porous layer is between 10 microns to 200 microns. In some embodiments, the average thickness of the first porous layer is greater than or equal to 10 microns, greater than or equal to 50 microns, greater than or equal to 75 microns, greater than or equal to 100 microns, greater than or equal to 150 microns, greater than or equal to 175 microns, greater than or equal to 200 microns. In some embodiments, the average thickness of the first porous layer is less than or equal to 200 microns, less than or equal to 175 microns, less than or equal to 150 microns, less than or equal to 100 microns, less than or equal to 75 microns, less than or equal to 50 microns, less than or equal to 10 microns. In some embodiments, the average thickness of a second porous layer is between 0 microns to 200 microns. Combinations of the above-referenced ranges are also possible (e.g., greater than or equal to 10 microns and less than or equal to 200 microns). Other ranges are also possible.

In some embodiments, a first porous layer comprises a porous structure (e.g., a braided, woven, and/or knitted structure) with an average density of between about 50 g/m² and 300 g/m². In some embodiments, the average density of the first porous layer is greater than or equal to 50 g/m², greater than or equal to 100 g/m², greater than or equal to 150 g/m², greater than or equal to 200 g/m², greater than or equal to 250 g/m², greater than or equal to 300 g/m². In some embodiments, the average density of the first porous layer is less than or equal to 300 g/m², less than or equal to 250 g/m², less than or equal to 200 g/m², less than or equal to 150 g/m², less than or equal to 100 g/m², and less than or equal to 50 g/m². Combinations of the above-referenced ranges are also possible (e.g., greater than or equal to 50 g/m² and less than or equal to 300 g/m²). Other ranges are also possible.

In some embodiments, the average pore size of a second porous layer is between about 0.01 mm and 5 mm. In some embodiments, the average pore size of the second layer is greater than or equal to 0.01 mm, greater than or equal to 0.05 mm, greater than or equal to 0.1 mm, greater than or equal to 0.5 mm, greater than or equal to 1.0 mm, greater than or equal to 1.5 mm, greater than or equal to 2 mm, greater than or equal to 3.0 mm, greater than or equal to 4 mm, and greater than or equal to 5 mm. In some embodiments, the average pore size of the second layer is less than or equal to 5.0 mm, less than or equal to 4 mm, less than or equal to 3 mm, less than or equal to 2 mm, less than or equal to 1.5 mm, less than or equal to 1.0 mm, less than or equal to 0.5 mm, less than or equal to 0.1 mm, less than or equal to 0.05 mm, and less than or equal to 0.01 mm. Combinations of the above-referenced ranges are also possible (e.g., greater than or equal to 0.01 mm and less than or equal to 5 mm). Other ranges are also possible.

In some embodiments, the average thickness of the second porous layer is greater than or equal to 0 microns, greater than or equal to 5 microns, greater than or equal to 10 microns, greater than or equal to 50 microns, greater than or equal to 75 microns, greater than or equal to 100 microns, greater than or equal to 150 microns, greater than or equal to 175 microns, greater than or equal to 200 microns. In some embodiments, the average thickness of the second porous layer is less than or equal to 200 microns, less than or equal to 175 microns, less than or equal to 150 microns, less than or equal to 100 microns, less than or equal to 75 microns, less than or equal to 50 microns, less than or equal to 10 microns, less than or equal to 5 microns, and less than or equal to 0 microns. Combinations of the above-referenced ranges are also possible (e.g., greater than or equal to 5 microns and less than or equal to 200 microns). Other ranges are also possible.

In some embodiments, the average density of the second porous layer is between 0 g/m² and 300 g/m². In some embodiments, the average density of the second porous layer is greater than or equal to 0 g/m², greater than or equal to 25 g/m², greater than or equal to 50 g/m², greater than or equal to 100 g/m², greater than or equal to 150 g/m², greater than or equal to 200 g/m², greater than or equal to 250 g/m², greater than or equal to 300 g/m². In some embodiments, the average density of the second porous layer is less than or equal to 300 g/m², less than or equal to 250 g/m², less than or equal to 200 g/m², less than or equal to 150 g/m², less than or equal to 100 g/m², less than or equal to 50 g/m², less than or equal to 25 g/m², and less than or equal to 0 g/m\hat{ }2. Combinations of the above-referenced ranges are also possible (e.g., greater than or equal to 0 g/m² and less than or equal to 300 g/m²). Other ranges are also possible.

In some embodiments, a prosthetic aortic implant comprises an expandable support structure comprising a first portion comprising a nonporous layer. As used herein, nonporous refers to a material or structure with no openings or openings sufficiently small to prevent blood flow within the ranges of physiological pressures. In some embodiments, the nonporous layer may comprise any suitable nonporous material (e.g., a biological material, a bioabsorbable material, a polymeric material, etc.) formed into any suitable geometry (e.g., sheets, films, hollow tubes, etc.) using any technique known to those of skill in the art. For example, in some embodiments, the nonporous layer may comprise a nonporous polymer film (e.g., polymeric balloon) formed from a medical grade thermoplastic (e.g., polyethylene, polypropylene, polymethyl methacrylate, polyvinyl chloride, polyamide, polycarbonate, ABS, etc.,) by extrusion (e.g., tubing extrusion, blow film extrusion, sheet film extrusion, over jacket extrusion, etc.), pultrusion, solvent-casting, or the like. In other embodiments, the nonporous material (e.g., woven nylon) may be provided over, within, or embedded inside a frame structure (e.g., a second expandable reinforcement structure).

In some embodiments, the nonporous layer may comprise a thin film (e.g., a balloon) configured to expand in response to physiological pressures (e.g., 20-200 mmHg). Exemplary thin films may be formed into a plurality of shapes, such as a conical shapes, square shapes, spherical shapes, conical/square shapes, conical/spherical shapes, long spherical shapes, tapered shapes, dog bone shapes, etc., In some embodiments, the nonporous layer may be configured to expand due to a blood hydrostatic pressure created by blood flowing through an intraluminal space formed between the nonporous layer and an adjacent first porous layer, wherein upon expansion, the nonporous layer applies a radially outward force to the ascending aorta thereby forming a seal against the internal surface of the aorta and/or anchoring the prosthetic aortic implant to the native aorta. Those of ordinary skill will appreciate that placing the nonporous layer across at least a portion of a dissection may form a seal around at least a portion of the dissection, thus preventing blood from flowing through the dissection. In some embodiments, the nonporous layer may have a suitable average oxygen permeability.

In some embodiments, a porous layer (e.g., a first porous layer and/or second porous layer) and/or a nonporous layer may comprise a coating, for example, to prevent plugging of the porous layer by adhesion of blood products (e.g., plasma, platelets, coagulation factors, red blood cells, white blood cells, hormones, proteins, fats, vitamins, etc.). In some embodiments, the coating may comprise grafting one or more hydrophilic polymers (e.g., polyethylene glycol), zwitterionic polymers (e.g., trimethylamine-n-oxide), and/or hydrophobic polymers (e.g., perfluorocarbons) to one or more surfaces of the layer (e.g., polymer and/or metallic surfaces). Those of ordinary skill in the art will appreciate that grafting perfluorocarbon like moieties to the surface may render the layer superhydrophobic, whereas grafting PEG-like and TMAO-like moieties to the surface may render the layer super hydrophilic, both of which are generally known to prevent adhesion of blood products to porous and nonporous substrates. In other embodiments, the coating may comprise increasing the surface hydrophilicity of the layer, for example, by oxygen plasma etching and/or layer by layer deposition of oppositely charged polymers (e.g., poly-L-lysine and polyacrylic acid).

According to some embodiments, the systems and methods described herein are compatible with one or more therapeutic, diagnostic, and/or enhancement agents, such as drugs, nutrients, microorganisms, in vivo sensors, and tracers. In some embodiments, the active substance, is a therapeutic, nutraceutical, prophylactic or diagnostic agent. While much of the specification describes the use of therapeutic agents, other agents listed herein are also possible.

Agents can include, but are not limited to, any synthetic or naturally-occurring biologically active compound or composition of matter which, when administered to a subject (e.g., a human or nonhuman animal), induces a desired pharmacologic, immunogenic, and/or physiologic effect by local and/or systemic action. For example, useful or potentially useful within the context of certain embodiments are compounds or chemicals traditionally regarded as drugs, vaccines, and biopharmaceuticals, Certain such agents may include molecules such as proteins, peptides, hormones, nucleic acids, gene constructs, etc., for use in therapeutic, diagnostic, and/or enhancement areas, including, but not limited to medical or veterinary treatment, prevention, diagnosis, and/or mitigation of disease or illness (e.g., HMG co-A reductase inhibitors (statins) like rosuvastatin, nonsteroidal anti-inflammatory drugs like meloxicam, selective serotonin reuptake inhibitors like escitalopram, blood thinning agents like clopidogrel, steroids like prednisone, antipsychotics like aripiprazole and risperidone, analgesics like buprenorphine, antagonists like naloxone, montelukast, and memantine, cardiac glycosides like digoxin, alpha blockers like tamsulosin, cholesterol absorption inhibitors like ezetimibe, metabolites like colchicine, antihistamines like loratadine and cetirizine, opioids like loperamide, proton-pump inhibitors like omeprazole, anti(retro)viral agents like entecavir, dolutegravir, rilpivirine, and cabotegravir, antibiotics like doxycycline, ciprofloxacin, and azithromycin, anti-malarial agents, and synthroid/levothyroxine); substance abuse treatment (e.g., methadone and varenicline); family planning (e.g., hormonal contraception); performance enhancement (e.g., stimulants like caffeine); and nutrition and supplements (e.g., protein, folic acid, calcium, iodine, iron, zinc, thiamine, niacin, vitamin C, vitamin D, and other vitamin or mineral supplements).

In certain embodiments, the active substance is one or more specific therapeutic agents. As used herein, the term “therapeutic agent” or also referred to as a “drug” refers to an agent that is administered to a subject to treat a disease, disorder, or other clinically recognized condition, or for prophylactic purposes, and has a clinically significant effect on the body of the subject to treat and/or prevent the disease, disorder, or condition. Listings of examples of known therapeutic agents can be found, for example, in the United States Pharmacopeia (USP), Goodman and Gilman's The Pharmacological Basis of Therapeutics, 10th Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17th ed. (1999), or the 18th ed (2006) following its publication, Mark H. Beers and Robert Berkow (eds.), Merck Publishing Group, or, in the case of animals, The Merck Veterinary Manual, 9th ed., Kahn, C.A. (ed.), Merck Publishing Group, 2005; and “Approved Drug Products with Therapeutic Equivalence and Evaluations,” published by the United States Food and Drug Administration (F.D.A.) (the “Orange Book”). Examples of drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. In certain embodiments, the therapeutic agent is a small molecule. Exemplary classes of therapeutic agents include, but are not limited to, analgesics, anti-analgesics, anti-inflammatory drugs, antipyretics, antidepressants, antiepileptics, antipsychotic agents, neuroprotective agents, anti-proliferatives, such as anti-cancer agents, antihistamines, antimigraine drugs, hormones, prostaglandins, antimicrobials (including antibiotics, antifungals, antivirals, antiparasitics), antimuscarinics, anxioltyics, bacteriostatics, immunosuppressant agents, sedatives, hypnotics, antipsychotics, bronchodilators, anti-asthma drugs, cardiovascular drugs, anesthetics, anti-coagulants, inhibitors of an enzyme, steroidal agents, steroidal or non-steroidal anti-inflammatory agents, corticosteroids, dopaminergics, electrolytes, muscle relaxants, nutritional agents, vitamins, parasympathomimetics, stimulants, anorectics and anti-narcoleptics. Nutraceuticals can also be incorporated into the drug delivery device. These may be vitamins, supplements such as calcium or biotin, or natural ingredients such as plant extracts or phytohormones.

In some embodiments, the therapeutic compound may comprise an antimicrobial compound, for example, to prevent infection following implantation of the device within a subject. In some embodiments, the antimicrobial compound comprises a penicillin. Non-limiting examples include penicillin V, penicillin G, amoxicillin, amoxicillin/clavulonate, ampicillin, nafcillin, oxacillin, dicloxacillin, piperacillin, pipercillin/tazobactam, and the like. In some embodiments, the antimicrobial compound comprises a macrolide. Examples include, but are not limited to, azithromycin, clarithromycin, fidaxomicin, erythromycin, telithromycin, and the like. In some embodiments, the antimicrobial compound comprises a cephalosporin. Examples include, but are not limited to, cefacetril, cefradin, cefroxadin, cefaloglycin, cefaclor, cefalexin, cefadroxil, cefatrizin, cefazedon, cefapirin, ceftezol, cefazolin, cefazaflur, cefalotin, cefaloridin, cefalonium, and the like. In some embodiments, the antimicrobial compound comprises a fluoroquinolone. Examples include balofloxacin, grepafloxacin, levofloxacin, pazufloxacin, sparfloxacin, temafloxacin, clinafloxacin, gatifloxacin, moxifloxacin, sitafloxacin, prulifloxacin, besifloxacin, delafloxacin, and the like. In some embodiments, the antimicrobial compound comprises a beta-lactam. Examples include penams, carbapenams, clavams, penems, carbapenems, cephems, carbacephems, oxacephems, monobactams, and the like. Combinations are also possible (e.g., the coating may comprise a penicillin and a beta-lactam or a fluoroquinolone and a cephalosporin, etc.).

In another embodiment, the therapeutic agent is an immunosuppressive agent. Exemplary immunosuppressive agents include glucocorticoids, cytostatics (such as alkylating agents, antimetabolites, and cytotoxic antibodies), antibodies (such as those directed against T-cell recepotors or Il-2 receptors), drugs acting on immunophilins (such as cyclosporine, tacrolimus, and sirolimus) and other drugs (such as interferons, opioids, TNF binding proteins, mycophenolate, and other small molecules such as fingolimod).

In some embodiments, a therapeutic compound may comprise an anti-proliferative compound to prevent, for example, a surrounding tissue from growing inside a conduit defined by an implantable structure (e.g., to prevent a device from becoming plugged). Exemplary embodiments of anti-proliferative compounds include mycophenolate mofetil, mycophenolate sodium, azathioprine, sirolimus, paclitaxel, etc.

In some embodiments, a prosthetic aortic graft comprises an expandable support structure. In some embodiments, the expandable support structure may be preformed with a curvature to conform to an aortic arch of a native aorta. In some embodiments, the expandable support structure comprises a first portion comprising a first porous layer positioned within a nonporous layer. In some embodiments, the first portion is configured to be positioned within an ascending portion of a native aorta, for example, from a brachiocephalic trunk to an aortic root. In some embodiments, the expandable support structure further comprises a second portion comprising a second porous layer sized and configured to be positioned within a descending portion of the native aorta. In some embodiments, the second portion may be configured to substantially cover the expandable support structure from the descending aorta to a brachiocephalic trunk (e.g., covering the branched vessels of the aortic arch). In this way, according to some embodiments, the second portion may further anchor the implant to the native aorta while permitting blood flow from within the expandable support structure (e.g., from the left ventricle of a heart) through the second porous layer and into the carotid arteries and/or subclavian arteries (e.g., positioning the second portion into the descending aorta does not obstruct blood flow). In other embodiments, the second portion may be configured to substantially cover the first portion, comprising the nonporous layer, such that the expandable support structure extends from a proximal end from within the ascending aorta to a distal end within the descending aorta.

The expandable support structure may comprise a cylindrical coil or wire frame that can comprise a sinusoidal wave pattern, Z-shape or zig-zag pattern. The expandable support structure can be configured to extend from the descending aorta to the ascending aorta and curve along with the curvature of the aortic arch when expanded within the aorta. In some embodiments, the distal end of the expandable support structure may comprise an expandable structure as described above. In some embodiments, the expandable structure can comprise an expandable trilobe structure that can be configured to be positioned within the aortic root of a patient and apply radial force to the sinuses of the aortic root. In some embodiments, the expandable structure may comprise a sinusoidal wave structure that may be configured to be positioned within the sinotubular junction and apply radial force to the sinotubular junction. In some embodiments, one or more portions or components of the prosthetic implant can be formed from fabric, metal, polymer or a biological tissue, and may be made of any of the materials described above. The first portion may be sized such that it is capable of reaching a diameter just slightly below that of the native ascending aorta (e.g., a maximum diameter of about 35 mm) when fully expanded with the expandable support structure inside. In other implementations, the first portion can have resting diameter of 35 mm and an expanded diameter of 40 mm such that it could be expanded by the support structure to contact the inner most wall of the native descending aorta. Those of ordinary skill in the art would understand that the resting diameter and expanded diameter provided above are for illustrative purposes only and that other resting diameters and expanded diameters are also possible, those of ordinary skill capable of selecting suitable resting diameters and expanded diameters based upon the teachings of this specification. The material of the first portion may be flexible enough to accommodate the curvature of the aortic arch. In some implementations, the entire length of first portion may be non-porous or the level of porosity may vary throughout the length of first portion. As described above, the porosity of the first portion could be configured, in some cases, to allow blood to flow into the carotid and subclavian arteries of the patient. The expandable support structure provides hoop strength and radial force beyond that of the portion, and serves to enhance the apposition of the portion against the intima, in some embodiments.

The second portion may be provided over the first portion 1814 may be configured to contact the site of the aortic dissection and the aortic wall adjacent to the false lumen. In some embodiments, the length of the second portion may be less than the length of the first portion. The second portion can extend along one of the lobes of the trilobe structure, e.g., the lobe positioned in the non-coronary aortic sinus, and the other two lobes remain uncovered so that blood may flow through the coronary ostia. The second portion can be formed from fabric, metal, polymer or a biological tissue, including any of the materials that may be utilized for the first portion.

In some embodiments, both ends of the second portion may be sealed to the first portion and the second portion may be configured to expand like a balloon when blood flows through the implant. For example, blood may flow through the first portion and expand the second portion such that there is space between the first portion and the second portion. The expanded diameter of the second portion may be larger than the diameter of the first portion (e.g., 45 mm versus 35 mm). The second portion may remain inflated against the aortic wall such that the second portion applies radial force to the aortic dissection site to seal the entry tear prevent blood from flowing into the false lumen. In some instances, an additional (third) portion that is non-porous may be disposed between the first porous portion and the second non-porous portion such that the third portion provides for a one-way valve that allows blood to enter the space between portions and but prevents it from exiting. This could be accomplished by laser cutting or otherwise creating gills, slots or flaps in the third portion that can open into the space during systole when blood pressure is highest but close against the first porous portion when that pressure is reduced during diastole preventing the blood from exiting.

In another embodiment, the aortic dissection implant can comprise a single portion that extends from the distal end to the proximal end of the expandable support structure. The single portion can comprise an inflatable non-porous section and a porous section proximal to the inflatable non-porous section.

The skilled artisan will understand that a prosthetic implant as disclosed herein may be used to treat any suitable disease or condition known to the skilled artisan. For example, in some embodiments, the implant is configured to treat an aortic dissection. In some cases, the aortic dissection is a Type A aortic dissection. Treatment of other conditions is also contemplated herein, according to other embodiments. For example, in some embodiments, the implant is configured to treat an intramural hematoma. Additionally, or alternatively, the implants disclosed herein may be used to treat a thoracic aortic aneurysm.

In some aspects, the disclosure relates to one or more of the following exemplary embodiments, although the disclosure is not so limited:

Embodiment 1. A prosthetic aortic implant, comprising: an expandable support structure having a first portion sized and configured to be positioned within an ascending portion of a native aorta, and a second portion, wherein the first portion is configured to apply radial force to an aortic root of the aorta when expanded; the first portion comprising: a non-porous layer adjacent a first porous layer and configured to contact an outer wall of the native aorta, an expandable anchoring structure located at a proximal end of the first portion sized and configured to engage an aortic root of the native aorta, and the second portion comprising a second porous layer; wherein the expandable anchoring structure comprises one or more backstop elements sized and configured to engage a native leaflet of the native aorta; and wherein the proximal end of the first portion is sized and configured to receive an aortic valve implant.

Embodiment 2. A prosthetic aortic implant as in Embodiment 1, wherein the proximal end of the first portion is in contact with but not directly adhered and/or grafted to the native aorta upon deployment in the native aorta.

Embodiment 3. A prosthetic aortic implant as in any preceding Embodiment, wherein the proximal end of the first portion comprises a prosthetic aortic valve frame.

Embodiment 4. A prosthetic aortic implant as in any preceding Embodiment, wherein the prosthetic aortic valve frame comprises a bridge valve.

Embodiment 5. A prosthetic aortic implant as in any preceding Embodiment, wherein the prosthetic aortic valve frame comprises a destination valve.

Embodiment 6. A prosthetic aortic implant as in any preceding Embodiment, wherein the prosthetic aortic valve frame is configured to receive the aortic valve implant.

Embodiment 7. A prosthetic aortic implant as in any preceding Embodiment, wherein the prosthetic aortic valve frame does not comprise a valve.

Embodiment 8. A prosthetic aortic implant as in any preceding Embodiment, wherein the aortic valve frame reversibly receives the aortic valve implant.

Embodiment 9. A prosthetic aortic implant as in any preceding Embodiment, wherein the aortic valve frame irreversibly receives the aortic valve implant.

Embodiment 10. The prosthetic aortic implant as in any preceding Embodiment, wherein a proximal end of the prosthetic aortic valve frame is flared and extends into a left ventricular outflow track (LVOT), thereby at least partially anchoring the prosthetic aortic implant to the native aorta.

Embodiment 11. A prosthetic aortic implant as in any preceding Embodiment, wherein the expandable anchoring structure comprises an expandable trilobe structure.

Embodiment 12. A prosthetic aortic implant as in any preceding Embodiment, wherein at least a portion of the expandable trilobe structure applies a radially outward force to an aortic sinus thereby anchoring the prosthetic aortic implant to the native aorta.

Embodiment 13. The prosthetic aortic implant as in any preceding Embodiment, wherein the expandable trilobe structure further comprises a trilobe structure comprising three lobes, wherein each of the three lobes is sized and configured to conform to a curvature of the aortic sinus when the expanded anchoring structure is expanded.

Embodiment 14. The prosthetic aortic implant as in any preceding Embodiment, wherein each of the three lobes comprises one or more apex at a distal end of the expandable trilobe structure configured to be positioned adjacent to an aortic valve annulus of the patient.

Embodiment 15. The prosthetic aortic implant as in any preceding Embodiment, wherein the trilobe structure is configured to expand to engage an inner wall of the aortic sinus separately from expansion of the first portion, and the prosthetic aortic implant is configured to sequentially deploy the trilobe structure before the first portion.

Embodiment 16. A prosthetic aortic implant as in any preceding Embodiment, wherein the one or more native leaflets are secured between the proximal end of the first portion and the one or more backstop elements of the expandable anchoring structure thereby at least partially anchoring the prosthetic aortic implant to the native aorta.

Embodiment 17. A prosthetic aortic implant as in any preceding Embodiment, wherein the one or more backstop elements of the expandable anchoring structure are sized and configured to prevent one or more of the native leaflets from blocking flow into one or more coronary arteries by ensuring the one or more native leaflets cannot expand beyond the backstops and block one or more ostia of a right coronary artery and/or left coronary artery.

Embodiment 18. The prosthetic aortic implant as in any preceding Embodiment, wherein the expandable anchoring structure engages one or more aortic sinuses thereby anchoring the prosthetic aortic implant and/or promoting a seal at within the region just above a sinotubular junction in the native aorta.

Embodiment 19. The prosthetic aortic implant as in any preceding Embodiment, wherein the one or more expandable anchoring structures, configured to extend within a left and a right aortic sinus, is at least partially uncovered such that the left and right coronary ostia remain uncovered by the prosthetic aortic implant when in use.

Embodiment 20. The prosthetic aortic implant as in any preceding Embodiment, wherein the expandable anchoring structure further comprises a metallic wire frame.

Embodiment 21. The prosthetic aortic implant as in any preceding Embodiment, wherein the first porous layer has an average areal density of between 50-300 g/m\hat{ }2

Embodiment 22. The prosthetic aortic implant as in any preceding Embodiment, wherein the first porous layer has an average pore size of between 0.01-2.00 MM.

Embodiment 23. The prosthetic aortic implant as in any preceding Embodiment, wherein the first porous layer has a thickness of between 10-200 microns.

Embodiment 24. The prosthetic aortic implant as in any preceding Embodiment, wherein the first porous layer comprises a first coating.

Embodiment 25. The prosthetic aortic implant as in any preceding Embodiment, wherein the first prevents adhesion of at least one or more components of blood.

Embodiment 26. The prosthetic aortic implant as in any preceding Embodiment, wherein the coating comprises a therapeutic.

Embodiment 27. The prosthetic aortic implant as in any preceding Embodiment, wherein the non-porous layer is configured to expand and, upon expansion, apply a radially outward force to the ascending aorta thereby forming a seal against the internal surface of the aorta and/or anchoring the prosthetic aortic implant to the native aorta.

Embodiment 28. The prosthetic aortic implant as in any preceding Embodiment, wherein the non-porous layer expands due to a blood hydrostatic pressure created by blood flowing through an intraluminal space formed between the non-porous layer and the first porous layer.

Embodiment 29. The prosthetic aortic implant as in any preceding Embodiment, wherein the non-porous layer contacts the inner wall of the native aorta between a brachiocephalic trunk and the sinotubular junction of the native aorta.

Embodiment 30. The prosthetic aortic implant as in any preceding Embodiment, wherein the non-porous layer is configured to be positioned across at least a portion of a dissection.

Embodiment 31. The prosthetic aortic implant as in any preceding Embodiment, wherein the non-porous layer is configured to seal around at least a portion of the dissection.

Embodiment 32. The prosthetic aortic implant as in any preceding Embodiment, wherein the non-porous layer is configured to prevent blood flow through the dissection.

Embodiment 33. The prosthetic aortic implant as in any preceding Embodiment, wherein the non-porous layer comprises an opening to allow blood to flow from within the expandable support structure, through the opening, and into the carotid and the subclavian arteries of the native aorta.

Embodiment 34. The prosthetic aortic implant as in any preceding Embodiment, wherein the non-porous layer comprises a second coating.

Embodiment 35. The prosthetic aortic implant as in any preceding Embodiment, wherein the second coating prevents adhesion of at least one or more component of blood.

Embodiment 36. The prosthetic aortic implant as in any preceding Embodiment, wherein the second coating comprises a therapeutic agent.

Embodiment 37. The prosthetic aortic implant as in any preceding Embodiment, wherein the second porous layer has an areal density of between 0-300 g/m\hat{ }2

Embodiment 38. The prosthetic aortic implant as in any preceding Embodiment, wherein the second porous layer has an average 0.01-5.00 MM.

Embodiment 39. The prosthetic aortic implant as in any preceding Embodiment, wherein the second porous layer has a thickness of between 0-200 microns.

Embodiment 40. The prosthetic aortic implant as in any preceding Embodiment, wherein the second porous layer comprises a third coating.

Embodiment 41. The prosthetic aortic implant as in any preceding Embodiment, wherein the third coating prevents adhesion of at least one or more components of blood to the second porous layer.

Embodiment 42. The prosthetic aortic implant as in any preceding Embodiment, wherein the third coating comprises a therapeutic compound.

Embodiment 43. The prosthetic aortic implant as in any preceding Embodiment, wherein the first portion comprises a metallic frame that extends continuously from the proximal end to a distal end of the first portion and that is continuous with the metallic frame of the expandable anchoring structure.

Embodiment 44. The prosthetic aortic implant as in any preceding Embodiment, wherein the first portion and the expandable anchoring structure comprise separate frames.

Embodiment 45. The prosthetic aortic implant as in any preceding Embodiment, wherein the first portion and the expandable anchoring structure are formed from a single continuous wire.

Embodiment 46. The prosthetic aortic implant as in any preceding Embodiment, wherein the second portion is sized and configured to be positioned within a descending portion of the native aorta

Embodiment 47. The prosthetic aortic implant as in any preceding Embodiment, wherein the second portion is configured to permit blood flow from within the expandable support structure, through the second porous layer, and into carotid arteries and/or subclavian arteries of the native aorta.

Embodiment 48. The prosthetic aortic implant as in any preceding Embodiment, wherein the second portion comprising the second porous layer is configured to substantially cover the expandable support structure from the descending aorta to a brachiocephalic trunk of the native aorta and the first portion comprising the non-porous layer partially covering the second portion and configured to engage a wall of the ascending aorta on opposite sides of a tear of the dissection.

Embodiment 49. The prosthetic aortic implant as in any preceding Embodiment, wherein the second portion comprises a metallic frame that extends continuously from a proximal end to a distal end of the second portion and that is continuous with the metallic frame of the expandable anchoring structure.

Embodiment 50. The prosthetic aortic implant as in any preceding Embodiment, wherein the second portion and the expandable anchoring structure comprise separate frames.

Embodiment 51. The prosthetic aortic implant as in any preceding Embodiment, wherein the first portion, the expandable anchoring structure, and the second portion comprise separate frames.

Embodiment 52. The prosthetic aortic implant as in any preceding Embodiment, wherein the first portion, the expandable anchoring structure, and the second portion are formed from a single continuous wire.

Embodiment 53. The prosthetic aortic implant as in any preceding Embodiment, wherein the second portion and the expandable anchoring structure are formed from a single continuous wire.

Embodiment 54. The prosthetic aortic implant as in any preceding Embodiment, wherein the expandable support structure is pre-formed with a curvature to conform to an aortic arch of the native aorta.

Embodiment 55. A system, comprising: a prosthetic aortic valve; and a prosthetic aortic implant comprising a proximal end sized and configured to receive the prosthetic aortic valve, wherein: the prosthetic aortic implant comprises a first portion sized and configured to be positioned within an ascending portion of a native aorta, one or more expandable anchoring structures, and a second portion sized and configured to be positioned within a descending portion of the native aorta, and wherein the expandable anchoring structure is sized and configured to apply radial force to an aortic root of the aorta when expanded.

Embodiment 56. The system as in Embodiment 55, wherein the prosthetic aortic implant is in contact with but not directly adhered and/or grafted to a native aorta upon deployment in the native aorta.

Embodiment 57. The system as in any preceding Embodiment, wherein the one or more expandable anchoring structures comprises a backstop element to engage one or more native leaflets of a native aortic valve

Embodiment 58. The system as in any preceding Embodiment, wherein the second portion comprises a second porous layer.

Embodiment 59. The system as in any preceding Embodiment, wherein the first portion comprises a non-porous layer adjacent a first porous layer and configured to contact an outer wall of the native aorta.

Embodiment 60. The system as in any preceding Embodiment, wherein the proximal end further comprises a prosthetic aortic valve frame.

Embodiment 61. The system as in any preceding Embodiment, wherein the prosthetic aortic valve frame is sized and configured to receive the prosthetic aortic valve

Embodiment 62. The system as in any preceding Embodiment, wherein the prosthetic aortic valve frame is not adhered to the native aorta.

Embodiment 63. The system as in any preceding Embodiment, wherein the prosthetic aortic valve is a bridge valve.

Embodiment 64. The system as in any preceding Embodiment, wherein the prosthetic aortic valve is a destination valve.

Embodiment 65. The system as in any preceding Embodiment, wherein the prosthetic aortic valve is a TAVI.

Embodiment 66. The systems as in any preceding Embodiment, wherein the prosthetic aortic valve is a unitary valve.

Embodiment 67. The system as in any preceding Embodiment, wherein the prosthetic aortic valve prevents regurgitation.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, e.g., elements that are conjunctively present in some cases and disjunctively present in other cases. Multiple elements listed with “and/or” should be construed in the same fashion, e.g., “one or more” of the elements so conjoined. Other elements may optionally be present other than the elements specifically identified by the “and/or” Embodiment, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, e.g., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (e.g., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, e.g., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Any terms as used herein related to shape, orientation, alignment, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter. Examples of such terms related to shape, orientation, and/or geometric relationship include, but are not limited to terms descriptive of: shape—such as, round, square, gomboc, circular/circle, rectangular/rectangle, triangular/triangle, cylindrical/cylinder, elliptical/ellipse, (n)polygonal/(n)polygon, etc.; angular orientation—such as perpendicular, orthogonal, parallel, vertical, horizontal, collinear, etc.; contour and/or trajectory—such as, plane/planar, coplanar, hemispherical, semi-hemispherical, line/linear, hyperbolic, parabolic, flat, curved, straight, arcuate, sinusoidal, tangent/tangential, etc.; direction—such as, north, south, east, west, etc.; surface and/or bulk material properties and/or spatial/temporal resolution and/or distribution—such as, smooth, reflective, transparent, clear, opaque, rigid, impermeable, uniform(ly), inert, non-wettable, insoluble, steady, invariant, constant, homogeneous, etc.; as well as many others that would be apparent to those skilled in the relevant arts. As one example, a fabricated article that would described herein as being “square” would not require such article to have faces or sides that are perfectly planar or linear and that intersect at angles of exactly 90 degrees (indeed, such an article can only exist as a mathematical abstraction), but rather, the shape of such article should be interpreted as approximating a “square,” as defined mathematically, to an extent typically achievable and achieved for the recited fabrication technique as would be understood by those skilled in the art or as specifically described. As another example, two or more fabricated articles that would described herein as being “aligned” would not require such articles to have faces or sides that are perfectly aligned (indeed, such an article can only exist as a mathematical abstraction), but rather, the arrangement of such articles should be interpreted as approximating “aligned,” as defined mathematically, to an extent typically achievable and achieved for the recited fabrication technique as would be understood by those skilled in the art or as specifically described.

1. A prosthetic aortic implant, comprising:
an expandable support structure having a first portion sized and configured to be positioned within an ascending portion of a native aorta, and a second portion, wherein the first portion is configured to apply radial force to an aortic root of the aorta when expanded;
the first portion comprising:
a non-porous layer adjacent a first porous layer and configured to contact an outer wall of the native aorta,
an expandable anchoring structure located at a proximal end of the first portion sized and configured to engage an aortic root of the native aorta, and
the second portion comprising a second porous layer;
wherein the expandable anchoring structure comprises one or more backstop elements sized and configured to engage a native leaflet of the native aorta; and
wherein the proximal end of the first portion is sized and configured to receive an aortic valve implant.

2-9. (canceled)

10. The prosthetic aortic implant as in claim 1 further comprising a prosthetic aortic valve frame, wherein a proximal end of the prosthetic aortic valve frame is flared and extends into a left ventricular outflow track (LVOT), thereby at least partially anchoring the prosthetic aortic implant to the native aorta.

11. The prosthetic aortic implant as in claim 1, wherein the expandable anchoring structure comprises an expandable trilobe structure, wherein at least a portion of the expandable trilobe structure applies a radially outward force to an aortic sinus thereby anchoring the prosthetic aortic implant to the native aorta.

12. (canceled)

13. The prosthetic aortic implant as in claim 11, wherein the expandable trilobe structure further comprises a trilobe structure comprising three lobes, wherein each of the three lobes is sized and configured to conform to a curvature of the aortic sinus when the expanded anchoring structure is expanded.

14. The prosthetic aortic implant as in claim 13, wherein each of the three lobes comprises one or more apex at a distal end of the expandable trilobe structure configured to be positioned adjacent to an aortic valve annulus of a patient.

15. The prosthetic aortic implant as in claim 11, wherein the expandable trilobe structure is configured to expand to engage an inner wall of the aortic sinus separately from expansion of the first portion, and the prosthetic aortic implant is configured to sequentially deploy the trilobe structure before the first portion.

16. The prosthetic aortic implant as in claim 1, wherein the one or more native leaflets are secured between the proximal end of the first portion and the one or more backstop elements of the expandable anchoring structure thereby at least partially anchoring the prosthetic aortic implant to the native aorta.

17. The prosthetic aortic implant as in claim 1, wherein the one or more backstop elements of the expandable anchoring structure are sized and configured to prevent one or more of the native leaflets from blocking flow into one or more coronary arteries by ensuring the one or more native leaflets cannot expand beyond the backstops and block one or more ostia of a right coronary artery and/or left coronary artery.

18. The prosthetic aortic implant as in claim 1, wherein the expandable anchoring structure engages one or more aortic sinuses thereby anchoring the prosthetic aortic implant and/or promoting a seal at within a region just above a sinotubular junction in the native aorta.

19. The prosthetic aortic implant as in claim 1, wherein the one or more expandable anchoring structures, configured to extend within a left and a right aortic sinus, is at least partially uncovered such that a left and right coronary ostia remain uncovered by the prosthetic aortic implant when in use.

20-26. (canceled)

27. The prosthetic aortic implant as in claim 1, wherein the non-porous layer is configured to expand and, upon expansion, apply a radially outward force to the ascending aorta thereby forming a seal against an internal surface of the aorta and/or anchoring the prosthetic aortic implant to the native aorta.

28. (canceled)

29. The prosthetic aortic implant as in claim 1, wherein the non-porous layer contacts an inner wall of the native aorta between a brachiocephalic trunk and a sinotubular junction of the native aorta.

30. The prosthetic aortic implant as in claim 1, wherein the non-porous layer is configured to be positioned across at least a portion of a dissection forming a seal around at least a portion of the dissection, thus preventing blood flow through the dissection.

31-32. (canceled)

33. The prosthetic aortic implant as in claim 1, wherein the non-porous layer comprises an opening to allow blood to flow from within the expandable support structure, through the opening, and into one or more carotid and subclavian arteries of the native aorta.

34-42. (canceled)

43. The prosthetic aortic implant as in claim 1, wherein the first portion comprises a metallic frame that extends continuously from the proximal end to a distal end of the first portion and that is continuous with the metallic frame of the expandable anchoring structure.

44. The prosthetic aortic implant as in claim 1, wherein the first portion and the expandable anchoring structure comprise separate frames.

45. The prosthetic aortic implant as in claim 1, wherein the first portion and the expandable anchoring structure are formed from a single continuous wire.

46. The prosthetic aortic implant as in claim 1, wherein the second portion is sized and configured to be positioned within a descending portion of the native aorta.

47. The prosthetic aortic implant as in claim 1, wherein the second portion is configured to permit blood flow from within the expandable support structure, through the second porous layer, and into carotid arteries and/or subclavian arteries of the native aorta.

48. The prosthetic aortic implant as in any preceding claim 1, wherein the second portion comprising the second porous layer is configured to substantially cover the expandable support structure from a descending aorta to a brachiocephalic trunk of the native aorta and the first portion comprising the non-porous layer partially covering the second portion and configured to engage a wall of the ascending aorta on opposite sides of a tear of a dissection.

49-54. (canceled)

55. A system, comprising:
a prosthetic aortic valve; and
a prosthetic aortic implant comprising a proximal end sized and configured to receive the prosthetic aortic valve,
wherein:
the prosthetic aortic implant comprises a first portion sized and configured to be positioned within an ascending portion of a native aorta, one or more expandable anchoring structures, and a second portion sized and configured to be positioned within a descending portion of the native aorta, and
wherein the expandable anchoring structure is sized and configured to apply radial force to an aortic root of the aorta when expanded.

56-67. (canceled)
